(19) United States
(12) Patent Application Publication
(10) Pub. No.: US 2019/0209884 A1
(43) Pub. Date: Jul. 11, 2019

(54) A PHYSIOTHERAPEUTIC BRACE AND METHODS THEREOF

(71) Applicants: Tal BUSKILA, Nahariya (IL); Oren COHEN, Moreshet (IL)

(72) Inventor: Tal BUSKILA, Nahariya (IL)

(21) Appl. No.: 16/326,972

(22) PCT Filed: Aug. 21, 2017
(86) PCT No.: PCT/IL2017/050932

§ 371 Date: Feb. 21, 2019

Related U.S. Application Data
(60) Provisional application No. 62/377,598, filed on Aug. 21, 2016.

(51) Int. Cl. A63B 21/04; A61F 5/01; A63B 21/00; A63B 21/055; A63B 23/035; A63B 23/04; A63B 23/08; A63B 23/12

(52) U.S. Cl. CPC A63B 21/0421; A61F 5/0127; A61F 5/0123; A61F 5/013; A63B 21/00061; A63B 21/0555; A63B 2022/0094; A63B 21/4017; A63B 21/4025; A63B 23/03508; A63B 23/0494; A63B 23/08; A63B 23/1281; A63B 21/4013

(57) ABSTRACT

The present invention discloses a modular brace, mountable on a joint, configured for exercising at least one muscle connected to, or otherwise affecting the function of, the joint, the brace comprising: lateral and medial rigid articulated cages, lateral and medial replaceable elastic members, and at least one joint-covering, replaceable elastic member. The lateral, medial, and at least one joint-covering, replaceable elastic members are configured to produce predetermined resistance force against muscles connected to the joint. The lateral and medial cages are configured for restriction of the joint's motion to the normal articulation of the joint. At least one joint-covering elastic member is adapted to simultaneously protect the joint from external insult while preventing pressure on the same.

### FIELD OF THE INVENTION

The present invention relates to modular brace (100), mountable on a joint and configured for physiotherapeutic exercising by producing predetermined resistance force against muscles connected to the joint. More specifically, the present invention pertains to a brace comprising lateral and medial rigid articulated cages (110, 120), lateral and medial replaceable elastic members (115, 125), each connected to end portions of said lateral and medial rigid articulated cages (110, 120), respectively and a at least one The joint-covering, replaceable elastic member (140), having a central opening (140A) configured for accommodation of the joint, connected to both said lateral and medial rigid articulated cages (110, 120).

### BACKGROUND OF THE INVENTION

The invention is in the field of continuous active motion (CAM) devices for use during the first phase of rehabilitation following a soft tissue surgical procedure or trauma. The goals of phase 1 rehabilitation are: control of post-operative pain, reduce inflammation, provide active motion in a specific plane of movement, and protect the healing repair or tissue. The CAM device constantly moves the joint through a controlled range of motion; the exact range is dependent upon the joint, but in most cases the range of motion is increased over time.

Knee pain is among the most commonly encountered orthopedic problems. While there are many causes of knee pain, most can be helped with some specific stretching and strengthening exercises. Even if surgery is necessary, rehabilitation exercises are certainly part of a recovery process.

The goals of knee rehabilitation are to strengthen the muscles that surround the knee while limiting the burden on the knee joint during the rehabilitation process. People who have stronger muscles surrounding the knee often have fewer problems with the joint. Weaker muscles create more burden for the knee joint by providing less support. Conversely, strong muscles of the leg better support and control the knee joint.

The muscles surrounding the knee include the quadriceps, hamstring, and calf muscles. The focus of most knee rehabilitation is on these muscles. When injuries occur, often these muscles become weaker and less supportive of the knee.

Exercises for the muscles that surround the knee include quadriceps strengthening exercises, hamstring strengthening exercises, and calf strengthening exercises.

Many patients rehab their knees by doing a select number of strengthening exercises a few times each day. But the fact is that just as critical as the overall strength, is the endurance of these muscles. Without endurance, these muscles will quickly fatigue.

Anterior cruciate ligament (ACL) is one of four knee ligaments that are critical to the stability of the knee joint. The ACL is made of tough fibrous material and functions to control excessive knee motion by limiting joint mobility.

One of the most common problems involving the knee joint is an anterior cruciate ligament injury or ACL tear. Of the four major knee ligaments of the knee, an ACL injury or rupture is the most debilitating knee ligament injury.

The best way to avoid ACL reconstructive surgery is to undertake a comprehensive ACL-Deficient Knee Rehabilitation Program that involves leg strengthening, proprioception and high level balance retraining, plus sport-specific agility and functional enhancement.

Prior art documents include U.S. Pat. No. 5,383,845, assigned to Nebolon, which discloses an orthopedic brace for supporting a joint has a plurality of compliant support components, including a hinged shell of stiffened elastic material and at least one supple support strap engaging the brace at two points on opposite sides of the hinge to stabilize the weakened joint throughout its range of motion. The shell has a pair of cuffs adapted to engage the body adjacent to the joint and connected to one another across a rotatable hinge by arms integrally extending from the cuffs. The support strap is adjustable in length enabling the user to modify the support strap tension, and consequently the degree of support the brace provides to the joint.

U.S. Pat. No. 8,257,293, granted to Ingimundarson et al., discloses a method for securing a knee brace having first and second frame members onto a leg includes the steps of: connecting a first end of a first stability strap to a buckle assembly and a second end to the first frame member so that the buckle assembly has locked and unlocked configurations. The method includes the additional steps of adjusting the length of the first stability strap; connecting a first end of a first force strap to the buckle assembly and a second end of the first force strap to the second frame member; adjusting the length of the first force strap; securing the buckle assembly to the first frame member in the unlocked configuration; and closing the buckle assembly against the first frame member so as to place the buckle assembly in the locked configuration.

U.S. Pat. No. 7,662,122, granted to Sterling, discloses an orthotic knee brace for treating unicompartmental osteoarthritis in a knee joint. The brace includes a fabric sleeve mounting and a bracing member removably positioned in the sleeve to provide a therapeutic force on the joint. The force is applied by a force dosimeter assembly that draws an incrementally adjustable amount of force on cables connected to the bracing member. The amount of force applied by the force dosimeter is detected by a force sensor, and is indicated by a detachable force indicator located on the fabric sleeve mounting. The foregoing features, while exemplified in the context of a knee brace, are adaptable to any orthotic or prosthetic device where it is desired to apply an adjustable amount of force to a point on the device to provide a therapeutic benefit, and to provide a real-time indication to the user of the amount of force being applied.

U.S. Pat. No. 4,606,542 (A), granted to David Segal, discloses an exerciser for a limb, such as a leg, comprising a brace hinged at the middle with upper padded members for snugly engaging the upper portion of a limb and lower padded members for snugly engaging the lower portion of a limb. Elastic hook-and-loop fasteners surround the members and the limb to firmly secure the members to the limb. A U-shaped portion extends above the center of the hinge and carries fulcrum assemblies on each leg of the U over which a resilient member rides. The ends of the resilient member are attached to upper studs at the far ends of the upper and lower assemblies. A second pair of resilient members may be connected between lower studs at the far end of each support assembly.

Chinese Patent #CN202169052(U) discloses a joint strength training device, which comprises a “sleeve I,” a “sleeve II,” and an elastic resistance band. Both sleeve I and sleeve II are made of elastic cloth capable of being bound on limbs, one end of the elastic resistance band is detachably connected onto sleeve I, and the other end of the elastic resistance band is detachably connected onto sleeve II. When the joint strength training device is used, sleeve I and sleeve II only need to be bound on large and small joints, ankles, wrists, knees, elbows and the like. Centralized and optimized muscular strength training and rehabilitation of damaged muscles can be performed for all parts of a body, and muscle and disease rehabilitation can be trained more scientifically. Simultaneously, the joint strength training device is simple in structure, high in adaptability, low in cost, and convenient to carry.

For a patient undergoing joint rehabilitation, there is a significant, practical advantage to having a way, outside of the physiotherapy clinic, of continuously developing muscle mass around the joint.

There is still a long felt and unmet need for a physiotherapeutic brace that will produce predetermined resistance force against muscles surrounding the treated joint, while supporting the joint and restricting the joint's motion to a given direction.

### SUMMARY OF THE INVENTION

It is thus one object of the present invention to disclose a modular brace (100), mountable on a joint, configured for exercising at least one muscle connected to, or otherwise affecting the function of, the joint, the brace (100) comprising:
a. lateral and medial rigid articulated cages (110, 120);
b. lateral and medial replaceable elastic members (115, 125), each connected to end portions of the lateral and medial rigid articulated cages (110, 120), respectively; and
c. at least one joint-covering, replaceable elastic member (140), having a central opening (140A) configured for accommodation of joint of the limb, connected to both the lateral and medial rigid articulated cages (110, 120);
   wherein the lateral, medial, and at least one joint-covering, replaceable elastic members (115, 125, 140) are configured to produce predetermined resistance force against muscles connected to the joint, while the lateral and medial cages (110, 120) are configured for restriction of the joint's motion to the normal articulation of the joint;
   further wherein the at least one joint-covering elastic member is adapted to simultaneously protect the joint from external insult while preventing pressure on the same.

It is another object of the present invention to disclose the modular brace mentioned above, wherein the brace is mountable on a knee.

It is another object of the present invention to disclose the modular brace mentioned above, wherein the brace is mountable on an elbow.

It is another object of the present invention to disclose the modular brace mentioned above, wherein the brace is mountable on an ankle.

It is another object of the present invention to disclose the modular brace mentioned above, wherein the lateral and medial replaceable elastic members (115) have a two-fold symmetrical axis (115B).

It is another object of the present invention to disclose the modular brace mentioned above, wherein the at least one joint-covering, replaceable elastic member (140) has a four-fold symmetrical axis (140B).

It is another object of the present invention to disclose the modular brace mentioned above, wherein dimensions of the central opening (140A) are configured for fixation of the joint.

It is another object of the present invention to disclose the modular brace mentioned above, wherein the lateral and medial replaceable elastic member are adjustable.

It is another object of the present invention to disclose the modular brace mentioned above, further comprising adjustable securing elements (131, 132), configured for mounting the brace (100) on the joint.

It is another object of the present invention to disclose the modular brace mentioned above, wherein the securing elements (131, 132) are padded.

It is another object of the present invention to disclose the modular brace mentioned above, wherein the securing elements (131, 132) comprise at least one fabric fastener (150) (, such as Velcro®).

It is another object of the present invention to disclose the modular brace mentioned above, wherein the lateral and medial cages (110, 120) are configured to simulate ligaments of the joint.

It is another object of the present invention to disclose the modular brace mentioned above, wherein the lateral, medial and at least one joint-covering, replaceable elastic members (115, 125, 140) are supplied in various degrees of elasticity for activating various degrees of resistance force.

It is another object of the present invention to disclose the modular brace mentioned above, wherein the at least one joint-covering, replaceable elastic member (140) comprises a ring (145) for the accommodation of the joint and four extensions or wings (141, 142, 143, 144) connecting the ring to both the lateral and medial rigid articulated cages (110, 120).

It is another object of the present invention to disclose the modular brace mentioned above, wherein the rigid articulated cages (110, 120) each comprise at least two elements (111, 112, 121, 122) and articulation means therebetween, the articulation means selected from a group consisting of:
a. pivot pins or ratchet pins (113, 123);
b. intermeshing gears; and
c. a pivoting ratchet assembly.

It is another object of the present invention to disclose the modular brace mentioned above, further comprising at least one pivot securing element (133) for stabilizing the location of the pivot pins or ratchet pins (113, 123) relative to the joint.

It is another object of the present invention to disclose the modular brace mentioned above, wherein the pivot securing element (133) comprises a strap configured opposite the joint-covering, replaceable elastic member (140) on the brace.

It is another object of the present invention to disclose the modular brace mentioned above, wherein the pivot securing element (133) is padded.

It is another object of the present invention to disclose the modular brace mentioned above, wherein the pivot securing element (133) comprises at least one fabric fastener (150) (, such as Velcro®).

It is another object of the present invention to disclose the modular brace mentioned above, wherein the pivot securing element (133) comprises at least one fastener from a group comprising: clip fasteners, lever fasteners, clamp fasteners, ratchet fasteners, and buckles.

It is another object of the present invention to disclose the modular brace mentioned above, further comprising a supporting stretch sleeve.

It is another object of the present invention to disclose the modular brace mentioned above, wherein the lateral and medial replaceable elastic members (115, 125) are made of a material selected from a group consisting of: natural rubber, synthetic rubber, silicone rubber, latex, mesh, web, and any combination thereof.

It is another object of the present invention to disclose the modular brace mentioned above, wherein the at least one joint-covering, replaceable elastic member (140) is made of a material selected from a group consisting of: natural rubber, synthetic rubber, silicone rubber, latex, mesh, web, and any combination thereof.

It is another object of the present invention to disclose the modular brace mentioned above, wherein the central opening (140A) is covered.

This is thus one object of the present invention to disclose a modular brace mentioned above, further comprising:
d. a sensor array (243) a sensor array configured to measure physiological data of said patient and said joint,
e. a digital processing means (241); said means configured to analyze said measured data, and presenting the input data by statistics graphs, further configured to store data in a server (248),
f. a communication means; said communication means (245) configured to receive said measured data and transmit data to at least one selected from the group consisting of a wireless module connection (244), a patient (242), a physician (246), a physiotherapist (246), a dedicated web-based application (247) and a combination thereof;

It is another object of the present invention to disclose the modular brace mentioned above, wherein said sensor array (243) of claim 111 configured for sensing physiological data from a group of parameters comprising:
a. the position and orientation of the brace relative to the joint; and
b. levels of individual straps deployed in said brace;
c. levels of current within muscle
d. levels of tension within muscles
e. levels of force of said muscles
f. range of movement of said joint
g. general movement of said patient

It is another object of the present invention to disclose the modular brace mentioned above, wherein said sensors (243) are selected from a group consisting an accelerometer, a sensor for movement measurement, a sensor for current measurement, a sensor for muscle tension and a combination thereof.

It is another object of the present invention to disclose the modular brace mentioned above, wherein said sensors (243) are interconnected within the wearable brace.

It is another object of the present invention to disclose the modular brace mentioned above, wherein said wireless connection is selected from the group consisting of: Bluetooth, antenna, wireless USB, wireless sensor networks, satellite communications, mobile data service and a combination thereof.

It is another object of the present invention to disclose the modular brace mentioned above, wherein said dedicated web application (247) further configured to intercommunicating with said patient (242) a group of parameters, said parameters comprising:
a. patient data analysis and statistics,
b. updated or modified treatment plans,
c. connection to other web users or other application users,
d. timely reminders for exercising.
e. quality of exercising

It is another object of the present invention to disclose the modular brace mentioned, wherein said dedicated web application (247) further configured to intercommunicating with Physiotherapist or Physician (246) a group of operations comprising:
a. collecting patient data transmitted by said sensors (243),
b. analyzing said measured data or interconnecting other web application, for further analyzing said data;
c. enabling said Physiotherapist or Physician (246) recommending modifying a treatment plan
d. monitoring technical status of said modular brace.

It is another object of the present invention to disclose the modular brace mentioned, further comprising a wireless communication means for transmitting said sensing information, wherein said data is transmitted to a dedicated web application (247), configured to functioning via a wired device or a wireless device, further wherein all data stored using local or cloud-based server (248).

It is another object of the present invention to disclose the modular brace mentioned above, wherein the sensor array comprises electronic fabric for measuring tension in a strand, or bundle of textile strands, in the straps.

It is another object of the present invention to disclose a method for providing exercise to at least one muscle connected to, or otherwise affecting the function of, a joint; the method comprising steps of:
a. providing a modular brace; the modular brace comprises:
   i. lateral and medial rigid articulated cages (110, 120);
   ii. lateral and medial replaceable elastic members (115, 125); and
   iii. at least one joint-covering, replaceable elastic member (140), having a central opening (140A);
b. connecting each of the lateral and medial replaceable elastic members (115, 125) to end portions of the lateral and medial rigid articulated cages (110, 120), respectively; and
c. accommodating the at least one joint-covering, replaceable elastic member (140) on the joint and connecting to both the lateral and medial rigid articulated cages (110, 120);
   thereby producing predetermined resistance force against muscles of the limb, while restricting the joint's motion to its normal articulation;
   thereby simultaneously protecting the joint from external insult and preventing pressure on the same by the at least one joint-covering, replaceable elastic member.

It is another object of the present invention to disclose the method mentioned above, additionally comprising the step of mounting the modular brace on a knee.

It is another object of the present invention to disclose the method mentioned above, additionally comprising the step of mounting the modular brace on an elbow.

It is another object of the present invention to disclose the method mentioned above, additionally comprising the step of mounting the modular brace on an ankle.

It is another object of the present invention to disclose the method mentioned above, additionally comprising the step of configuring the lateral and medial replaceable elastic members (115B) to each have a two-fold symmetrical axis.

It is another object of the present invention to disclose the method mentioned above, additionally comprising the step of configuring the at least one joint-covering, replaceable elastic member to have a four-fold symmetrical axis (140B).

It is another object of the present invention to disclose the method mentioned above, additionally comprising the step of configuring the dimensions of the central opening (140A) for the fixation of the joint.

It is another object of the present invention to disclose the method mentioned above, additionally comprising the step of configuring the lateral and medial replaceable elastic member to be adjustable.

It is another object of the present invention to disclose the method mentioned above, further comprising the step of providing adjustable securing elements (131, 132), configured for mounting the brace (100) on the joint.

It is another object of the present invention to disclose the method mentioned above, additionally comprising the step of configuring the securing elements (131, 132) to be padded.

It is another object of the present invention to disclose the method mentioned above, additionally comprising the step of configuring at least one of the securing elements (131, 132) to comprise a fabric fastener (150) (, such as Velcro®).

It is another object of the present invention to disclose the method mentioned above, additionally comprising the step of configuring the lateral and medial cages (110, 120) to simulate lateral and medial collateral ligaments of the joint.

It is another object of the present invention to disclose the method mentioned above, additionally comprising the step of configuring the lateral and medial cages (110, 120) to simulate radial and ulnar collateral ligaments of an elbow.

It is another object of the present invention to disclose the method mentioned above, further comprising the step of providing the lateral, medial and at least one joint-covering replaceable elastic members (115, 125, 140) in various degrees of elasticity for activating various degrees of resistance force.

It is another object of the present invention to disclose the method mentioned above, additionally comprising the step of configuring the at least one joint-covering, replaceable elastic member (140) to further comprise a ring (145) for the accommodation of the joint, and four extensions or wings (141, 142, 143, 144) connecting the ring to both the lateral and medial rigid articulated supporters (110, 120).

It is another object of the present invention to disclose the method mentioned above, additionally comprising the step of configuring the rigid articulated cages (110, 120) to each comprise at least two elements (111, 112, 121, 122) and articulation means therebetween, the articulation means selected from a group consisting of:
a. pivot pins or ratchet pins (113, 123);
b. intermeshing gears; and
c. a pivoting ratchet assembly.

It is another object of the present invention to disclose the method mentioned above, further comprising at least one pivot securing element (133) for stabilizing the location of the pivot pins or ratchet pins (113, 123) relative to the joint.

It is another object of the present invention to disclose the method mentioned above, wherein the pivot securing element (133) comprises a strap configured opposite the joint-covering, replaceable elastic member (140) on the brace.

It is another object of the present invention to disclose the method mentioned above, wherein the pivot securing element (133) is padded.

It is another object of the present invention to disclose the method mentioned above, wherein the pivot securing element (133) comprises at least one fabric fastener (150) (, such as Velcro®).

It is another object of the present invention to disclose the method mentioned above, wherein the pivot securing element (133) comprises at least one fastener from a group comprising: clip fasteners, lever fasteners, clamp fasteners, ratchet fasteners, and buckles.

It is another object of the present invention to disclose the method mentioned above, further comprising the step of providing the brace with a supporting stretch sleeve.

It is another object of the present invention to disclose the method mentioned above, additionally comprising the step of configuring the lateral and medial replaceable elastic cages (115, 125) to be made of a material selected from a group consisting of: natural rubber, synthetic rubber, silicone rubber, latex, mesh, web, and any combination thereof.

It is another object of the present invention to disclose the method mentioned above, additionally comprising the step of configuring the at least one joint-covering, replaceable elastic member (140) to be made of a material selected from a group consisting of: natural rubber, synthetic rubber, silicone rubber, latex, mesh, web, and any combination thereof.

It is another object of the present invention to disclose the method mentioned above, further comprising steps of providing a sensor array (243), a digital processing means (241), and a communication means (245); said sensory array (243) configured to measure physiological data of said patient and said joint; said digital processing means configured to analyze said measured data, further (245) stored in a server (248); said communication means (245) configured to receive said measured data and transmit data to at least one selected from the group consisting of a wireless module connection (244), a patient (242), a physician ((246), a physiotherapist (246), a dedicated web-based application (247) and a combination thereof.

It is another object of the present invention to disclose the method mentioned above, said sensor array (243) sensing physiological data from a group of parameters comprising:
a. the position and orientation of the brace relative to the joint; and
b. levels of individual straps deployed in said brace;
c. levels of current within muscle
d. levels of tension within muscles
e. levels of force of said muscles
f. range of movement of said joint
g. general movement of said patient

It is another object of the present invention to disclose the method mentioned above, wherein said sensors comprise at least one parameter from a group comprising an accelerometer, a sensor for movement measurement, a sensor for current measurement, a sensor for muscle tension and a combination thereof.

It is another object of the present invention to disclose the method mentioned above, wherein said sensors interconnecting within the wearable brace.

It is another object of the present invention to disclose the method mentioned above, wherein said wireless connection (244) comprise at least one parameter from a group comprising of: Bluetooth, antenna, wireless USB, wireless sensor networks, satellite communications, mobile data service and a combination thereof.

It is another object of the present invention to disclose the method mentioned above, wherein said dedicated web application (247) intercommunicating with said patient a group of parameters, said parameters comprising:
a. patient data analysis and statistics,
b. updated or modified treatment plans,
c. connection to other web users or application users,
d. timely reminders for exercising.
e. quality of exercising

It is another object of the present invention to disclose the method mentioned above, wherein said dedicated web application further intercommunicating with Physiotherapist or Physician (246) a group of operations, said operations comprising:
a. collecting patient data transmitted by said sensors,
b. analyzing said measured data or interconnecting other web application, for further analyzing said data;
c. enabling said Physiotherapist or Physician recommending modifying a treatment plan.
d. monitoring technical status of said modular brace.

It is another object of the present invention to disclose the method mentioned above,

wherein said modular brace further comprising a wireless communication means for transmitting said sensing information
wherein said data is transmitted to a dedicated web application, configured to
functioning via a wired device or a wireless device, further wherein all data stored using local or cloud-based server.

It is another object of the present invention to disclose the method mentioned above, further comprising the step of implementing the sensor array by means of electronic fabric; wherein the electronic fabric outputs tension data measured in a strand, or bundle of textile strands, in the straps.

It is another object of the present invention to disclose a set of replaceable elastic members for interconnecting lateral and medial rigid articulated cages (110, 120) of a modular brace which is mountable on a joint and configured for exercising at least one muscle connected to, or otherwise affecting the function of, the joint;

the replaceable elastic members (115, 125, 140) comprise at least one selected from a group comprising: lateral (115), medial (125), and joint-covering (140);
the set comprise a plurality of elastic members selected from a group consisting of: at least two lateral elastic members, at least two medial elastic members, at least two joint-covering elastic members;
wherein the replaceable elastic members (115, 125, 140) are configured to produce predetermined resistance force against muscles connected to the joint;
further wherein each of the components of the set is adapted to provide a different amount of the predetermined resistance force.

It is another object of the present invention to disclose the set of replaceable elastic members mentioned above, wherein the components of the set provide different amount of the predetermined resistance force by at least one selected from a group consisting of: differing in elasticity and differing in size.

It is another object of the present invention to disclose the set of replaceable elastic members mentioned above, wherein the lateral and medial cages (110, 120) are configured for restriction of the joint's motion to its normal articulation.

It is another object of the present invention to disclose the set of replaceable elastic members mentioned above, wherein each of the elastic members have a color according to the amount of predetermined resistance force provided by them.

It is another object of the present invention to disclose the set of replaceable elastic members mentioned above, wherein the lateral and the medial replaceable elastic members (115, 125), are adapted each to be connected to end portions of the lateral and medial rigid articulated cages (110, 120), respectively.

It is another object of the present invention to disclose the set of replaceable elastic members mentioned above, wherein the at least one joint-covering, replaceable elastic member (140), has a central opening (140A) configured for accommodating the joint.

It is another object of the present invention to disclose the set of replaceable elastic members mentioned above, wherein the at least one joint-covering, replaceable elastic member (140) is connected to both the lateral and medial rigid articulated cages (110, 120).

The set of claim 57, wherein the at least one joint-covering, replaceable elastic member (140) is adapted to simultaneously protect the joint from external insult while preventing pressure on the same.

It is another object of the present invention to disclose the set of replaceable elastic members mentioned above, wherein the brace is mountable on a joint selected from a group consisting of: knee, elbow, ankle, and any combination thereof.

It is another object of the present invention to disclose the set of replaceable elastic members mentioned above, wherein the replaceable elastic members are made of a material selected from a group consisting of: natural rubber, synthetic rubber, silicone rubber, latex, mesh, web, and any combination thereof.

It is another object of the present invention to disclose a joint-covering, replaceable elastic member (140) for accommodation of a joint; the at least one joint-covering, replaceable elastic member (140) is adapted to connect to lateral and medial rigid articulated cages (110, 120) of a modular brace (100) configured for exercising; wherein the at least one joint-covering, replaceable elastic member (140) has a central opening (140A) adapted to simultaneously protect the joint from external insult while preventing pressure on the same.

It is another object of the present invention to disclose a joint-covering, replaceable elastic member mentioned above, wherein the brace is mountable on a joint selected from a group consisting of: knee, elbow, ankle, and any combination thereof.

It is another object of the present invention to disclose a joint-covering, replaceable elastic member mentioned above, wherein the lateral and medial cages (110, 120) are configured for restriction of the joint's motion to its normal articulation.

It is another object of the present invention to disclose a joint-covering, replaceable elastic member mentioned above, wherein the frontal replaceable elastic member has a four-fold symmetrical axis (140B).

It is another object of the present invention to disclose a joint-covering, replaceable elastic member mentioned above, wherein dimensions of the central opening (140A) are configured for fixation of the joint.

It is another object of the present invention to disclose a joint-covering, replaceable elastic member mentioned above, wherein the frontal replaceable elastic members (140) is supplied in various degrees of elasticity thereby activating various degrees of resistance force.

It is another object of the present invention to disclose a joint-covering, replaceable elastic member mentioned above, wherein the frontal replaceable elastic member (140) comprises a ring (145) for the accommodation of the joint and four extensions or wings (141, 142, 143, 144) connecting the ring to both the lateral and medial rigid articulated cages (110, 120).

It is another object of the present invention to disclose a joint-covering, replaceable elastic member mentioned above, wherein the frontal replaceable elastic member (140) is made of a material selected from a group consisting of: natural rubber, synthetic rubber, silicone rubber, latex, mesh, web, and any combination thereof.

It is another object of the present invention to disclose a modular ankle brace (300), mountable on a foot configured for exercising at least one muscle connected to an ankle joint, comprising:
a. lateral and medial rigid articulated cages (110, 120) having each a central opening (113) configured for accommodation on the ankle;
b. lateral and medial replaceable elastic members (115, 125), each connected to end portions of the lateral and medial rigid articulated cages (110, 120), respectively;
c. a rear replaceable elastic member (140), configured for accommodation on a heel, connected to both the lateral and medial rigid articulated cages (110, 120);
   wherein the lateral, medial and rear replaceable elastic members (115, 125, 140) are configured to produce predetermined resistance force against muscles connected to the ankle, while the lateral and medial cages (110, 120) are configured for restriction of the ankle motion to its normal articulation;
   further wherein the lateral and medial rigid articulated cages are adapted to simultaneously protect the ankle from external insult while preventing pressure on the same.

It is another object of the present invention to disclose the modular ankle brace mentioned above, wherein the lateral and medial replaceable elastic members (115) have a two-fold symmetrical axis (115B).

It is another object of the present invention to disclose the modular ankle brace mentioned above, wherein the rear replaceable elastic member has a four-fold symmetrical axis (140B).

It is another object of the present invention to disclose the modular ankle brace mentioned above, wherein dimensions of the central opening (140A) are configured for fixation of the heel.

It is another object of the present invention to disclose the modular ankle brace mentioned above, wherein the lateral and medial replaceable elastic member are adjustable.

It is another object of the present invention to disclose the modular ankle brace mentioned above, further comprising adjustable securing elements (131, 132), configured for mounting the brace (100) on the ankle.

It is another object of the present invention to disclose the modular ankle brace mentioned above, wherein the securing elements (131, 132) are padded.

It is another object of the present invention to disclose the modular ankle brace mentioned above, wherein the securing elements (131, 132) comprise a fabric fastener (150).

It is another object of the present invention to disclose the modular ankle brace mentioned above, wherein the lateral and medial cages (110, 120) are configured to simulate ligaments of the ankle.

It is another object of the present invention to disclose the modular ankle brace mentioned above, wherein the lateral, medial and rear replaceable elastic members (115, 125, 140) are supplied in various degrees of elasticity thereby activating various degrees of resistance force.

It is another object of the present invention to disclose the modular ankle brace mentioned above, wherein the rear replaceable elastic member (140) comprises a ring (145) for the accommodation of the heel and four extensions or wings (141, 142, 143, 144) connecting the ring to both the lateral and medial rigid articulated cages (110, 120).

It is another object of the present invention to disclose the modular ankle brace mentioned above, wherein the rigid articulated cages (110, 120) each comprises at least two elements (111, 112, 121, 122) and articulation means there between, the articulation means selected from a group consisting of:
a. pivot pin or ratchet pin (113, 123);
b. intermeshing gears; and
c. pivoting ratchet assembly.

It is another object of the present invention to disclose the modular ankle brace mentioned above, further comprising a supporting stretch sleeve.

It is another object of the present invention to disclose the modular ankle brace mentioned above, wherein the lateral and medial replaceable elastic members (115, 125) are made of a material selected from a group consisting of: natural rubber, synthetic rubber, silicone rubber, latex, mesh, web, and any combination thereof.

It is another object of the present invention to disclose the modular ankle brace mentioned above, wherein the rear replaceable elastic member (140) is made of a material selected from a group consisting of: natural rubber, synthetic rubber, silicone rubber, latex, mesh, web, and any combination thereof.

It is another object of the present invention to disclose the modular ankle brace mentioned above, wherein the central opening (140A) is covered.

It is another object of the present invention to disclose a method for providing exercise to at least one muscle connected to, or otherwise affecting the function of, an ankle joint, comprising steps of:
a. providing:
   i. lateral and medial rigid articulated cages (110, 120) having each a central opening (140A); configured for accommodation on the ankle;
   ii. lateral and medial replaceable elastic members (115, 125); and,
   iii. a rear replaceable elastic member (140), having a central opening (140A); accommodating the lateral and medial rigid articulated cages on the ankle;
b. connecting the lateral and medial replaceable elastic members to end portions of the lateral and medial rigid articulated cages (110, 120), respectively; and
c. accommodating the central opening of the rear replaceable elastic member (140) on a heel and connecting the same to both the lateral and medial rigid articulated cages (110, 120);
   thereby producing predetermined resistance force against muscles of the ankle, while restricting the ankle's motion to its normal articulation;
   thereby simultaneously protecting the ankle from external insult while preventing pressure on the same by the lateral and medial rigid articulated cages.

It is another object of the present invention to disclose the method mentioned above, additionally comprising the step of configuring the lateral and medial replaceable elastic members (115B) to each have a two-fold symmetrical axis.

It is another object of the present invention to disclose the method mentioned above, additionally comprising the step of configuring the rear replaceable elastic member to have a four-fold symmetrical axis (140B).

It is another object of the present invention to disclose the method mentioned above, additionally comprising the step of configuring dimensions of the central opening (140A) for the fixation of the ankle.

It is another object of the present invention to disclose the method mentioned above, further comprising the step of providing adjustable securing elements (131, 132), configured for mounting the brace (100) on the foot.

It is another object of the present invention to disclose the method mentioned above, additionally comprising the step of configuring the securing elements (131, 132) to be padded.

It is another object of the present invention to disclose the method mentioned above, additionally comprising the step of configuring the securing elements (131, 132) to comprise a fabric (, such as Velcro®,) fastener (150).

It is another object of the present invention to disclose the method mentioned above, additionally comprising the step of configuring the lateral and medial cages (110, 120) to simulate lateral and medial collateral ligaments of the ankle.

It is another object of the present invention to disclose the method mentioned above, further comprising the step of providing the lateral, medial and rear replaceable elastic members (115, 125, 140) in various degrees of elasticity thereby for activating various resistance forces.

It is another object of the present invention to disclose the method mentioned above, additionally comprising the step of configuring the rear replaceable elastic member (140) to comprise a ring (145) for the accommodation of the ankle, and four extensions or wings (141, 142, 143, 144) connecting the ring to both the lateral and medial rigid articulated supporters (110, 120).

It is another object of the present invention to disclose the method mentioned above, additionally comprising the step of configuring the rigid articulated cages (110, 120) each comprises at least two elements (111, 112, 121, 122) and articulation means there between, the articulation means selected from a group consisting of:
a. pivot pin or ratchet pin (113, 123);
b. intermeshing gears; and
c. pivoting ratchet assembly.

It is another object of the present invention to disclose the method mentioned above, further comprising the step of providing the brace with a supporting stretch sleeve.

It is another object of the present invention to disclose the method mentioned above, wherein the lateral and medial replaceable elastic cages (115, 125) are made of a material selected from a group consisting of: natural rubber, synthetic rubber, silicone rubber, latex, mesh, web, and any combination thereof.

It is another object of the present invention to disclose the method mentioned above, wherein the rear replaceable elastic member (140) is made of a material selected from a group consisting of: natural rubber, synthetic rubber, silicone rubber, latex, mesh, web, and any combination thereof.

It is another object of the present invention to disclose a set of replaceable elastic members for interconnecting lateral and medial rigid articulated cages (110, 120) of a modular ankle brace (300) which is mountable on an ankle and configured for exercising at least one muscle connected to the same;

the replaceable elastic members (115, 125, 140) comprise at least one selected from a group comprising of: lateral (115), medial (125), and rear (140);
the set comprises a plurality of elastic members selected from a group consisting of: at least two lateral elastic members, at least two medial elastic members, at least two rear elastic members;
wherein the replaceable elastic members (115, 125, 140) are configured to produce predetermined resistance force against muscles connected to the ankle;
further wherein each of the components of the set is adapted to provide a different amount of the predetermined resistance force.

It is another object of the present invention to disclose the set of replaceable elastic members mentioned above, wherein the components of the set provide different amount of the predetermined resistance force by at least one selected from a group consisting of: differing in elasticity and differing in size.

It is another object of the present invention to disclose the set of replaceable elastic members mentioned above, wherein the lateral and medial cages (110, 120) are configured for restriction of the ankle's motion to its normal articulation.

It is another object of the present invention to disclose the set of replaceable elastic members mentioned above, wherein each of the elastic members have a color according to the amount of predetermined resistance force provided by them.

It is another object of the present invention to disclose the set of replaceable elastic members mentioned above, wherein the lateral and the medial replaceable elastic members (115, 125), are adapted each to be connected to end portions of the lateral and medial rigid articulated cages (110, 120), respectively.

It is another object of the present invention to disclose the set of replaceable elastic members mentioned above, wherein the rear replaceable elastic member (140), has a central opening (140A) configured for accommodating the heel.

It is another object of the present invention to disclose the set of replaceable elastic members mentioned above, wherein the rear replaceable elastic member (140) is connected to both the lateral and medial rigid articulated cages (110, 120).

It is another object of the present invention to disclose the set of replaceable elastic members mentioned above, wherein the replaceable elastic members are made of a material selected from a group consisting of: natural rubber, synthetic rubber, silicone rubber, latex, mesh, web, and any combination thereof.

### BRIEF DESCRIPTION OF THE DRAWINGS

In order to understand the invention and to see how it may be implemented in practice, a plurality of embodiments will now be described, by way of non-limiting example only, with reference to the accompanying drawings, in which:

FIG. 1 presents the knee's anatomy;

FIG. 2 presents the elbow's anatomy;

FIG. 3 presents the ankle's anatomy;

FIGS. 4A and 4B present illustrated, detailed views of the modular brace mounted on a knee;

FIG. 5 presents an illustrated view of the modular brace, with a rear, adjustable pivot securing strap, mounted on a knee;

FIGS. 6A, 6B, and 6C present illustrated views of the modular brace mounted on an elbow;

FIGS. 7A and 7B present illustrated views of the modular ankle brace mounted on an ankle;

FIG. 8 presents an illustrated view of the brace's replaceable elastic members;

FIG. 9 presents an illustrated view of a set of lateral, medial, and joint-covering, replaceable elastic members each exerting different amount of resistant force when attached to the brace;

FIG. 10 presents an illustrated view of the two-fold symmetry of the lateral and medial replaceable elastic member;

FIG. 11 presents an illustrated view of the four-fold symmetry of the at least one joint-covering, replaceable elastic member;

FIG. 12 presents an illustrated view of the location of the sensory array (243) interconnecting to lateral and medial rigid articulated cages (110, 120).

FIG. 13 presents a scheme of a system (240) for conducting a remote medical examination of patient and joint.

FIG. 14 presents a method of conducting a remote medical examination of patient and joint.

### DETAILED DESCRIPTION OF PREFERRED EMBODIMENTS

The following description is provided, alongside all chapters of the present invention, so as to enable any person skilled in the art to make use of the invention and sets forth the best modes contemplated by the inventor of carrying out this invention. Various modifications, however, are adapted to remain apparent to those skilled in the art, since the generic principles of the present invention have been defined specifically to provide a physiotherapeutic brace.

The term “articulated” used herein refers having two or more sections connected by a joint.

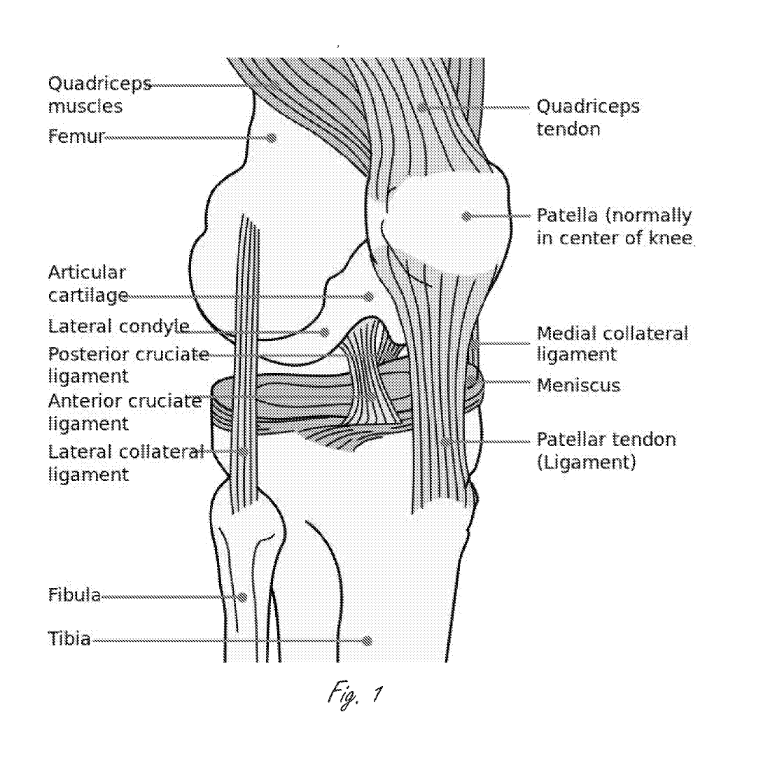

The term “knee” used herein refers to the joint that joins the thigh with the leg and consists of two articulations: one between the femur and tibia, and one between the femur and patella. It is the largest joint in the human body. The knee is a mobile trocho-ginglymus (a pivotal condylar joint), which permits flexion and extension as well as a slight internal and external rotation. FIG. 1 presents the knees anatomy.

The term “patella” used herein refers to the kneecap which is a thick, circular-triangular bone which articulates with the femur (thigh bone) and covers and protects the anterior articular surface of the knee joint. The patella can be observed in FIG. 1.

The term “elbow” used herein refers to the synovial hinge joint between the humerus in the upper arm and the radius and ulna in the forearm. Being a hinge joint, the only movements allowed by the elbow are flexion and extension of the joint and rotation of the radius. The range of motion of the elbow is limited by the olecranon of the ulna, so that the elbow can only extend to around 180 degrees. Flexion of the elbow is limited only by the compression soft tissues surrounding the joint. FIG. 2 presents the elbow's anatomy.

The term “olecranon” used herein refers to the large, thick, curved bony eminence of the forearm that projects behind the elbow. The olecranon can be observed in FIG. 2.

The central opening of the frontal elastic member is adapted to fit and protect the olecranon situated at the upper (proximal) end of the ulna bone.

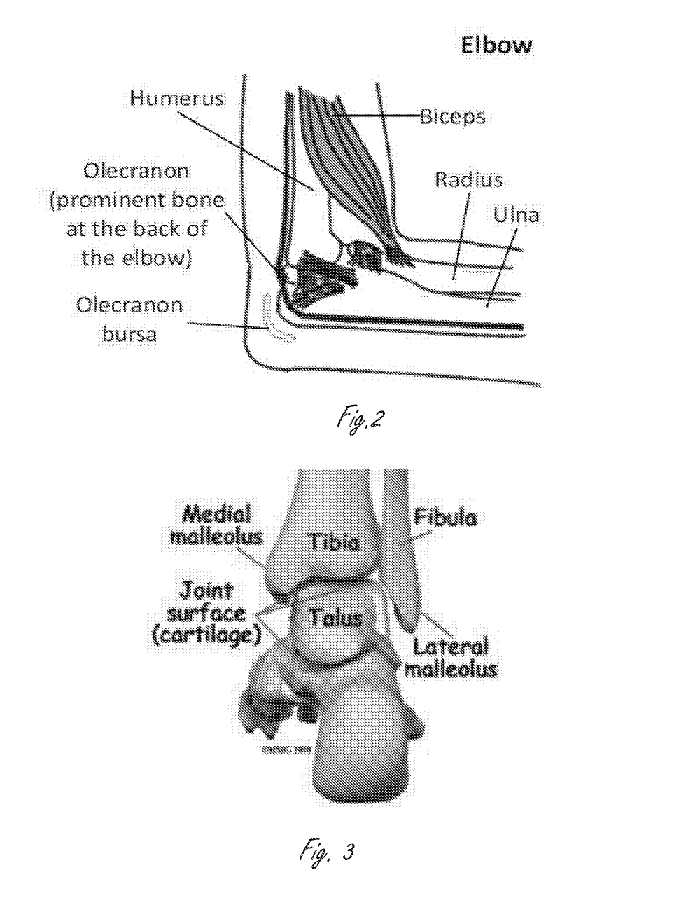

The term “ankle” used herein refers to the talocrural region, where the foot and the leg meet. The ankle includes three joints: the ankle joint proper or talocrural joint, the subtalar joint, and the Inferior tibiofibular joint. The movements produced at this joint are dorsiflexion and plantarflexion of the foot. The bony architecture of the ankle consists of three bones: the tibia, the fibula, and the talus. The articular surface of the tibia is referred to as the plafond. FIG. 3 presents the ankle's anatomy.

The term “malleolus” used herein refers to the bony prominence on each side of the ankle. The medial malleolus is the prominence on the inner side of the ankle, formed by the lower end of the tibia. The lateral malleolus is the prominence on the outer side of the ankle, formed by the lower end of the fibula. The malleolus can be observed in FIG. 3.

The present invention provides a modular brace (100), mountable on a joint configured for physiotherapeutic exercising, demonstrated in details at FIG. 1. The brace comprising: (a) lateral and medial rigid articulated cages (110, 120); (b) lateral and medial replaceable elastic members (115, 125), each connected to end portions of said lateral and medial rigid articulated cages (110, 120), respectively; (c) a at least one joint-covering, replaceable elastic member (140), having a central opening (140A) configured for accommodation of joint of said limb, connected to both said lateral and medial rigid articulated cages (110, 120); The central opening of the frontal elastic member prevents pressure on the joint (the patella, for instance) and also provides protection. The protection is provided by the elastic member according to its thickness. wherein said lateral, medial and at least one joint-covering, replaceable elastic members (115, 125, 140) are configured to produce predetermined resistance force against muscles connected to said joint, while said lateral and medial cages (110, 120) are configured for restriction of said joint's motion to a given direction; further wherein said at least one joint-covering, replaceable elastic member is adapted to simultaneously protect said joint from external insult while preventing pressure on the same.

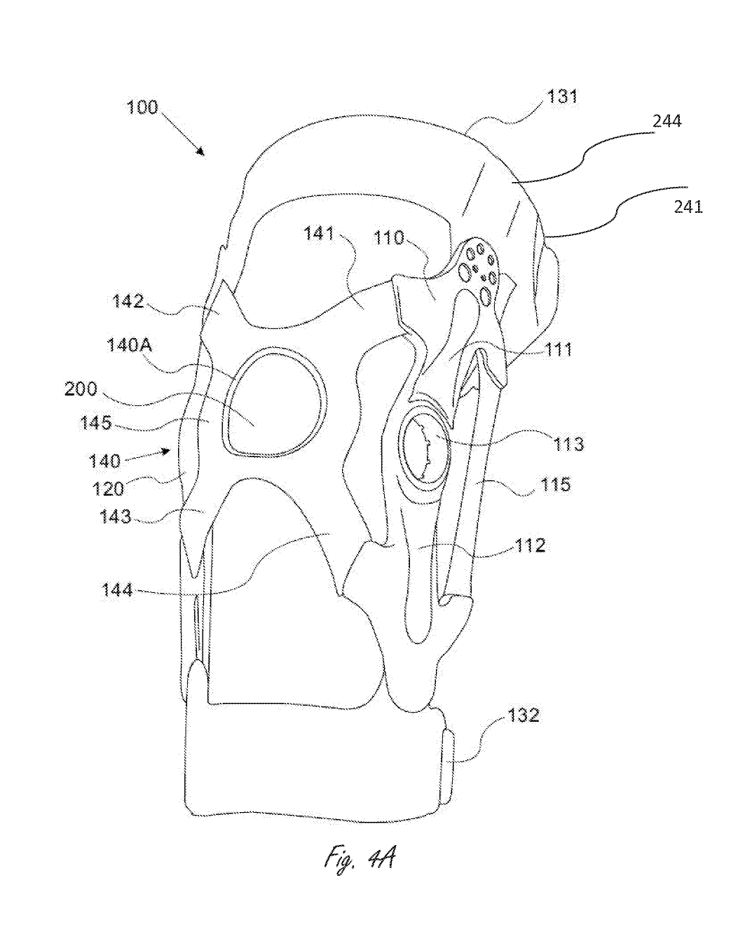

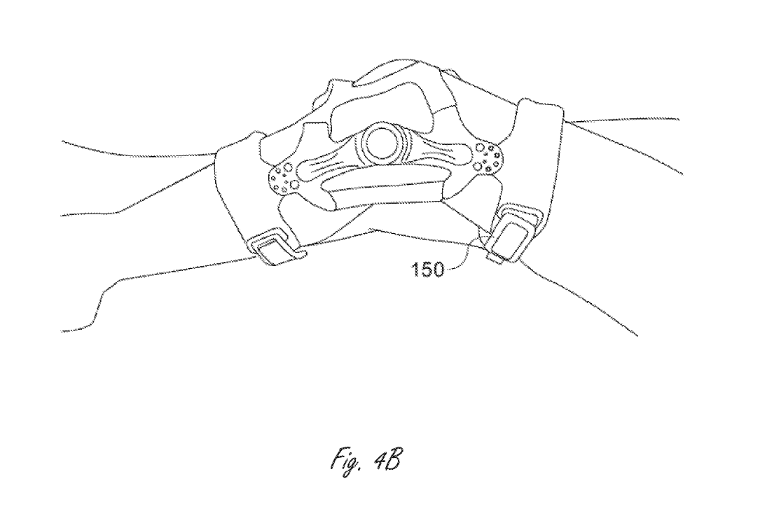

FIGS. 4A and 4B: presents an illustrated detailed view of the modular brace;

According to an embodiment of the invention, the brace (140) is mountable on a joint selected from a group consisting of: knee, elbow, ankle, and any combination thereof. FIG. 4 is an illustration of the brace mounted on the knee.

According to an embodiment of the invention, The modular brace of claim 2, wherein said at least one muscle is selected from a group consisting of Articularis genus, Sartorius, Quadriceps femoris, Rectus femoris, Vastus lateralis, Vastus intermedius, Vastus medialis, Biceps femoris, Semitendinosus, Semimembranosus, Gastrocnemius, Plantaris, Popliteus, Gracilis, and any combination thereof.

If the modular brace (100) is mounted on the knee then the opening of the joint-covering, replaceable elastic member is placed on the patella and provides protection and prevents pressure. FIGS. 4A and 4B present illustrated, detailed views of the modular brace mounted on a knee.

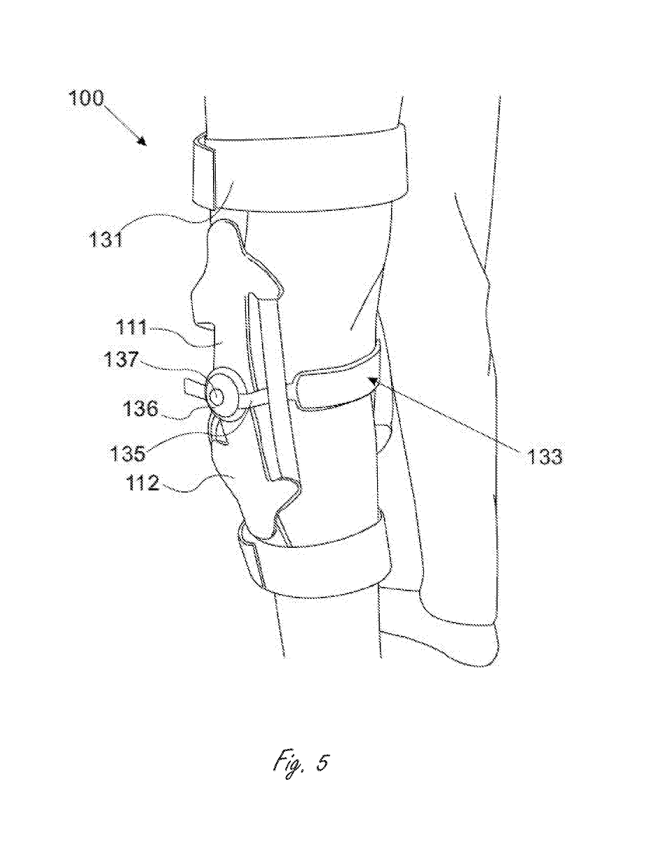

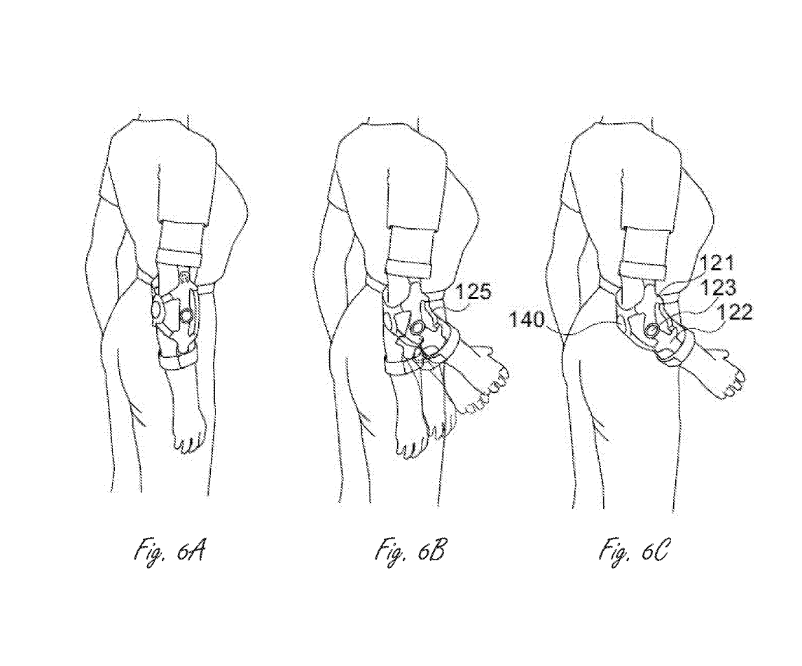

If the modular brace (100) is mounted on the elbow then the opening of the j joint-covering, replaceable elastic member is placed on the olecranon and provides protection and prevents pressure. FIGS. 6A, 6B, and 6C present an illustrated views of the modular brace mounted on an elbow.

The modular brace (100) designated for the ankle (not shown) will comprise two joint-covering, replaceable elastic members. One will be placed on the medial malleolus and the other on the lateral malleolus and therefore protects it and prevents pressure.

The present invention further provides a method for providing physiotherapeutic training to a limb's joint, comprising steps of: (a) providing: (i) lateral and medial rigid articulated cages (110, 120); (ii) lateral and medial replaceable elastic members (115, 125), each connected to end portions of said lateral and medial rigid articulated cages (110, 120), respectively; and (iii) a at least one joint-covering, replaceable elastic member (140), having a central opening (140A) configured for accommodating said joint, tensionally connected to both said lateral and medial rigid articulated cages (110, 120); (b) producing predetermined resistance force against muscles of said limb, while (c) restricting said joint's motion to a given direction; (d) simultaneously protecting said joint from external insult while preventing pressure on the same by said at least one joint-covering, replaceable elastic member.

The lateral and medial replaceable elastic members (115, 125) and the joint-covering, replaceable elastic member (140) are set in a stretched posture between the rigid articulated cages (110, 120) and any extension or flexion of the limb extends at least one of the elastic members (115, 125, 140) such that a resistance tension force is applied on the limb's muscles.

The present invention further provides a modular ankle brace (300), mountable on a foot configured for physiotherapeutic exercising of an ankle, comprising: (a) lateral and medial rigid articulated cages (110, 120) having each a central opening (113) configured for accommodation on said ankle; (b) lateral and medial replaceable elastic members (115, 125), each connected to end portions of said lateral and medial rigid articulated cages (110, 120), respectively; (c) a rear replaceable elastic member (140), configured for accommodation on a heel, connected to both said lateral and medial rigid articulated cages (110, 120); wherein said lateral, medial and rear replaceable elastic members (115, 125, 140) are configured to produce predetermined resistance force against muscles connected to said ankle, while said lateral and medial cages (110, 120) are configured for restriction of said ankle motion to its normal articulation; further wherein said lateral and medial rigid articulated cages are adapted to simultaneously protect said ankle from external insult while preventing pressure on the same.

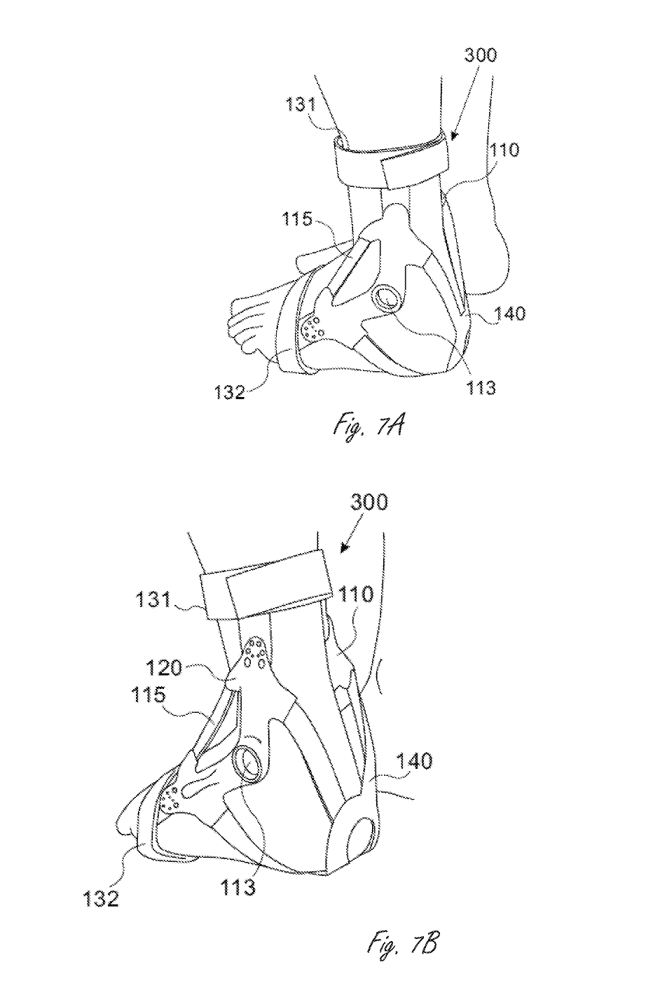

Reference is now made to FIGS. 7A and 7B. The modular ankle brace (300) differs from the modular brace (100) in that the rigid articulated cages (110, 120) are placed on the malleolus while the opening (113) is circling it. The rigid medial articulated cage is encircling the medial malleolus and the rigid lateral articulated cage is encircling the lateral malleolus and provides them protection as well as prevents pressure on them. The rear replaceable elastic member (140) is placed on the heel.

The present invention further provides a set of replaceable elastic members for interconnecting lateral and medial rigid articulated cages (110, 120) of a modular brace which is mountable on a joint and configured for physiotherapeutic exercising; the replaceable elastic members (115, 125, 140) comprise at least one selected from a group comprising of: lateral (115), medial (125), and joint-covering (140); the set comprise a plurality of elastic members selected from a group consisting of: at least two lateral elastic members, at least two medial elastic members, at least two joint-covering members; wherein said replaceable elastic members (115, 125, 140) are configured to produce predetermined resistance force against muscles connected to said joint; further wherein each of said components of said set is adapted to provide a different amount of said predetermined resistance force.

The medial, lateral and at least one joint-covering, replaceable elastic members may be supplied as a set, each providing a different amount of predetermined resistance force against muscles connected to said joint. Each degree of elasticity may be presented by a different color, thus enabling the user to decide which one to use. For example, the user will begin with the elastic member with the highest degree of elasticity and with progress will move gradually to the members with the lowest degree of elasticity. The user may also follow different instructions of a physiotherapist or physician to use the members of the different elasticity in any other order they will find suitable.

The different amount of resistance force can be achieved by changing size of the elastic members as shown in FIG. 9 or by changing the elasticity of the material from which they are made.

The present invention further provides a at least one joint-covering, replaceable elastic member (140) for accommodation of a joint; said a at least one joint-covering, replaceable elastic member (140) is adapted to connect to lateral and medial rigid articulated cages (110, 120) of a modular brace (100) configured for physiotherapeutic exercising; wherein said at least one joint-covering, replaceable elastic member (140) has a central opening (140A) adapted to simultaneously protect said joint from external insult while preventing pressure on the same.

According to an embodiment of the invention, the lateral and medial replaceable elastic members (115) have a two-fold symmetrical axis (115B).

Reference is now made to FIG. 10, which shows an illustrated view of the two fold symmetry of the lateral and medial replaceable elastic members. The symmetry enables simple securing of the lateral and medial cages on the brace with no directionality problems. The lateral and medial replaceable elastic member may also be identical in both their sides, therefore making it insignificant to which side is facing the skin and which side is facing the outer environment. These features will make the assembly of the members on the brace very simple without any consideration of directionality making it approachable even for elderly. It will also provide a quick way to replace those members which will induce compliance of the user.

Reference is now made to FIG. 11, which shows an illustrated view of the four fold symmetry (140B) of joint-covering, replaceable elastic member (140). The symmetry enables simple securing of the joint-covering, replaceable elastic member to the brace with no directionality problems. The joint-covering member may also be identical in both its sides, therefore making it insignificant to which side is facing the skin and which side is facing the outer environment. The lack of directionality makes the brace approachable even for elderly. It will also provide a quick way to replace those members which will induce compliance of users.

According to an embodiment of the invention, the dimensions of the central opening (140A) are configured for fixation of the joint for example the elbow, the ankle or the knee's patella.

According to an embodiment of the invention, the lateral and medial replaceable, elastic members are adjustable.

According to an embodiment of the invention, the brace further comprises two adjustable securing elements (131, 132), configured for mounting the brace (100) on the limb of the joint, for example on the arm around the elbow, as shown in FIGS. 6A, 6B, and 6C or on the leg around the knee, as demonstrated in FIG. 3. FIGS. 6A, 6B, and 6C further demonstrate the brace in use, showing flexed and extended positions of the arm.

According to an embodiment of the invention, the securing elements (131, 132) are padded, for comfortable use of the brace.

According to an embodiment of the invention, the securing elements (131, 132) comprise a fabric fastener such as Velcro® (150), configured for size adjustment to the user's measures.

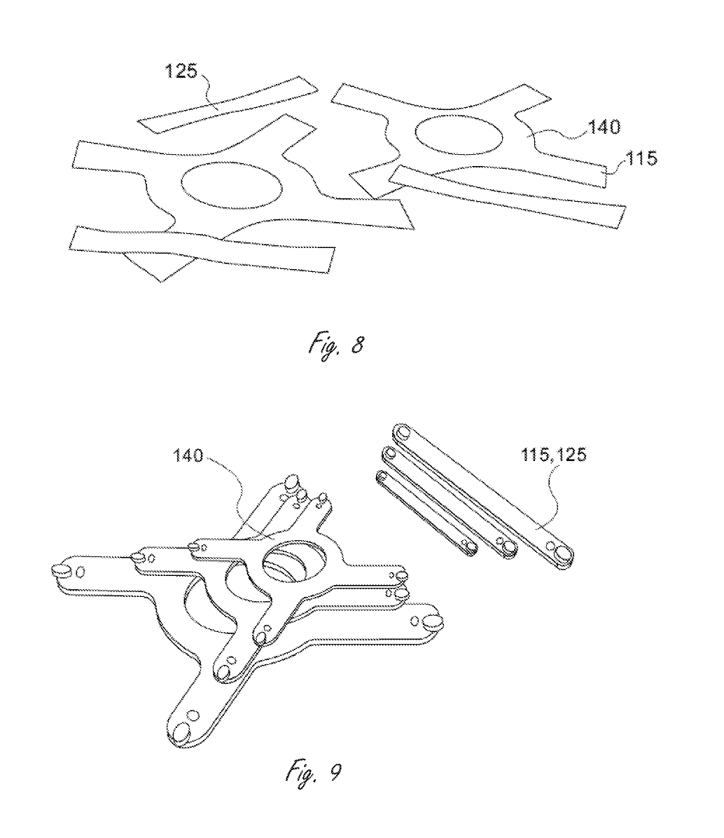

According to an embodiment of the invention, the lateral and medial cages (110, 120) are configured to simulate lateral and medial collateral ligaments of a knee, as demonstrated in prior art FIG. 8.

According to another embodiment of the invention, the lateral and medial cages (110, 120) are configured to simulate radial and ulnar collateral ligaments of an elbow.

According to an embodiment of the invention, the lateral, medial and at least one joint-covering, replaceable elastic members (115, 125, 140) are supplied in various degrees of elasticity thereby for activating various degrees of resistance force, as demonstrated in FIG. 4. According to another embodiment lateral and medial replaceable elastic members (115, 125) are similar.

According to an embodiment of the invention, the at least one joint-covering, replaceable elastic member (140) comprises a ring (145) for the accommodation of the joint and four extensions or wings (141, 142, 143, 144) connecting the ring to both the lateral and medial rigid articulated cages (110, 120), as demonstrated in FIG. 1.

According to an embodiment of the invention, the rigid articulated cages (110, 120) each comprise at least two elements (111, 112, 121, 122) and articulation means therebetween, said articulation means selected from a group consisting of:
(a) a pivot pin or ratchet pin (113);
(b) intermeshing gears;
(c) pivoting ratchet assembly, for example, male and female elements.

According to an embodiment of the invention, the modular brace further comprises at least one pivot securing element (133) for stabilizing the location of the pivot pins or ratchet pins (113, 123) relative to the joint.

According to an embodiment of the invention, the pivot securing element (133) comprises a strap configured opposite the joint-covering, replaceable elastic member (140) on the brace.

According to an embodiment of the invention, the pivot securing element (133) is padded.

According to an embodiment of the invention, the pivot securing element (133) comprises at least one fabric fastener (150) (, such as Velcro®).

According to an embodiment of the invention, the pivot securing element (133) comprises at least one fastener from a group comprising: clip fasteners, lever fasteners, clamp fasteners, ratchet fasteners, and buckles.

FIG. 5 presents a preferred embodiment of the modular brace (100) configured for the knee. A soft strap embodies the pivot securing element (133), securing the brace across the back of the knee, stabilizing the brace's pivots (113, 123) by countering the stress forces exerted on the pivots by the joint-covering, replaceable elastic member (140). Two plastic adjustment straps (135) are sewn as extensions to both sides of the soft strap pivot securing element (133). Each plastic adjustment strap is threaded through strap-locking mechanisms (136) located at the brace's lateral and medial pivots (113, 123). The strap-locking mechanisms are integrated with the pivots (113, 123) such that the mechanisms do not hinder the pivots' function of enabling free rotation of the brace's rigid upper parts (111, 121) relative to its lower parts (112, 122).

The plastic adjustment straps (135) are configured with notches or protruding ridges for enabling the straps (135) to be fastened with ratcheting strap-locking mechanisms (136). The plastic adjustment straps (135) are pulled through the strap-locking mechanisms (136) until the soft strap pivot securing element (133) is appropriately situated around the back of the knee and tightened. A central button (137) on each strap-locking mechanism (136) releases its fastened strap (135), for when the wearer removes or readjusts the brace.

The ratchet mechanism embodiment (136) does not limit the claim. The soft strap pivot securing element (133) may also be held in place by other fastening embodiments, including methods such as Velcro strap loops, tied laces, clasps, and buckles.

According to an embodiment of the invention, the brace further comprises a supporting stretch sleeve (not shown), also configured for thermal isolation.

According to an embodiment of the invention, the lateral and medial replaceable elastic members (115, 125) are made of a material selected from a group consisting of: natural rubber, synthetic rubber, silicone rubber, latex, mesh, web, and any combination thereof.

According to an embodiment of the invention, the at least one joint-covering, replaceable elastic member (140) is made of a material selected from a group consisting of: natural rubber, synthetic rubber, silicone rubber, latex, mesh, web, and any combination thereof.

According to an embodiment of the invention, the central opening (140A) is covered. The covering is not necessarily elastic and is made of different materials than the frontal member. The aim of the covering is to provide protection to the skin covering the joint. The cover is not meant to have any therapeutic effect.

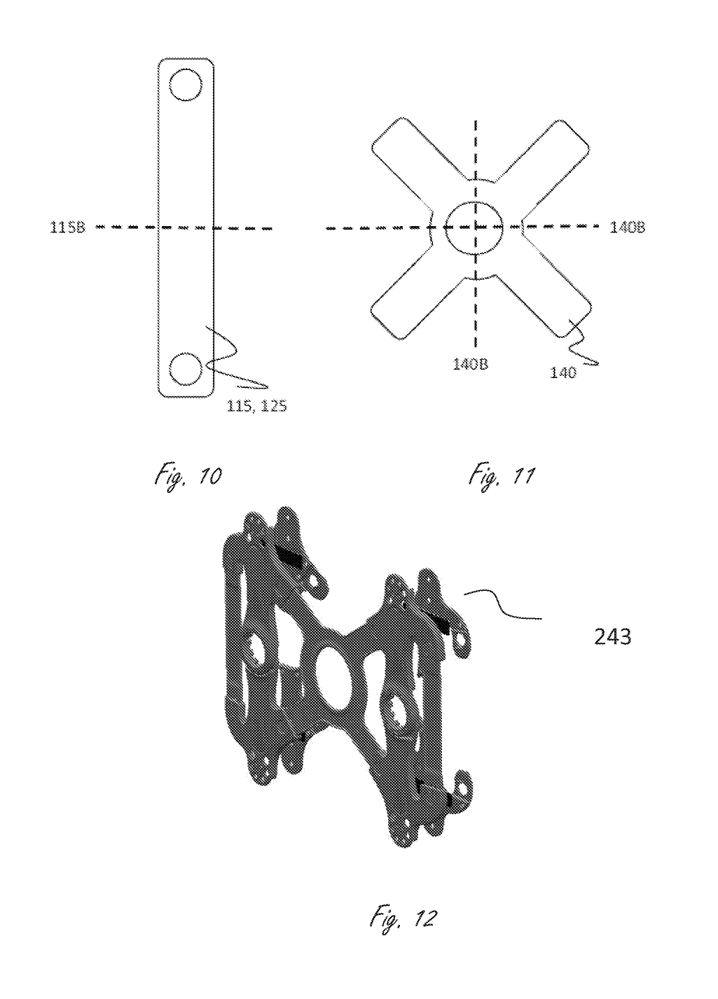

FIG. 12 presents the location of the sensory array (243) interconnecting to lateral and medial rigid articulated cages (110, 120).

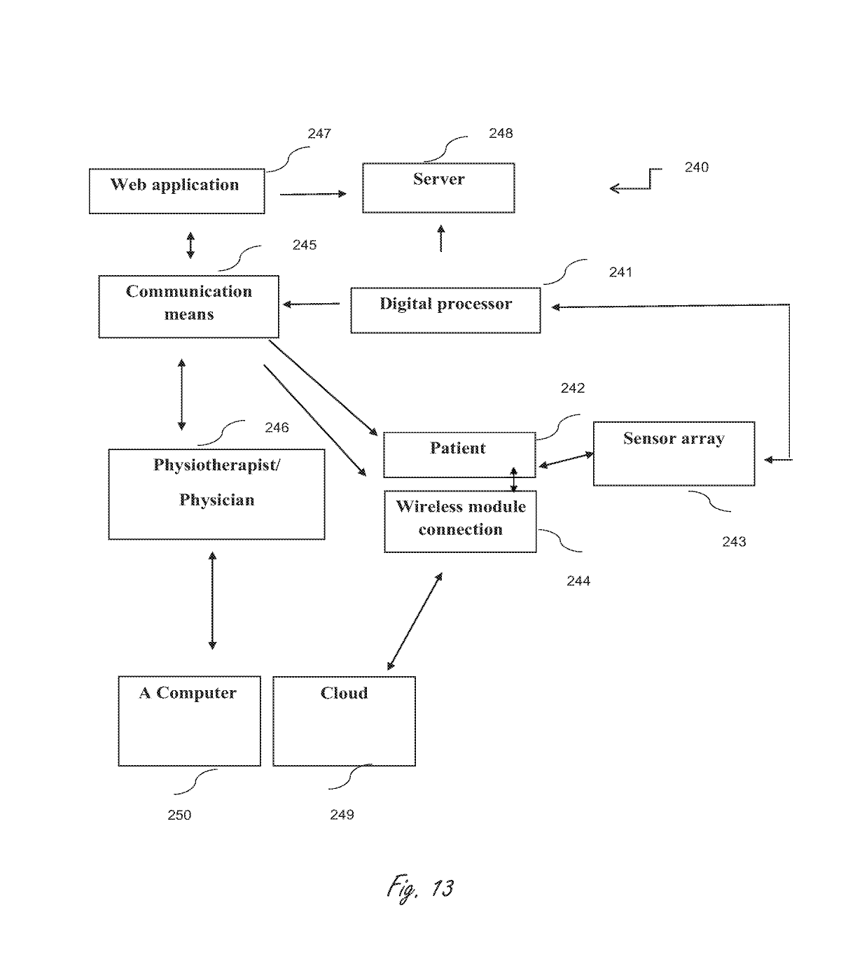

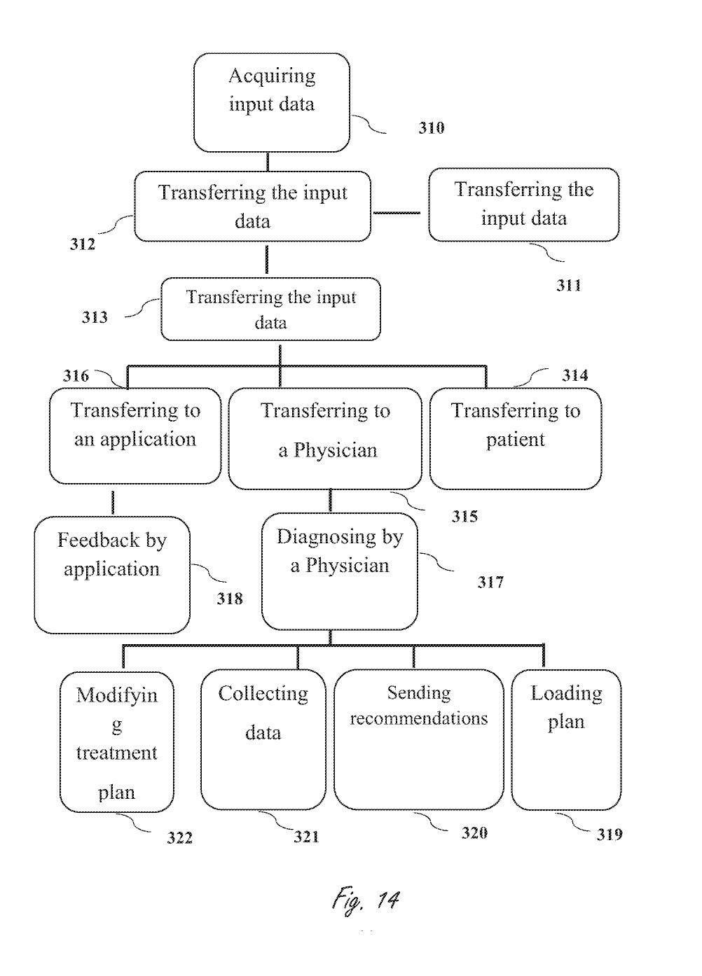

FIG. 13 presents the system of a remote medical examination of patient and joint

The system comprises a sensor array (243) interconnected within the wearable brace, configured to measure physiological data of said patient and said joint; a digital processing means (241) configured to analyze said measured data, and presenting the input data by statistics graphs, further configured to store data in a server (248); a communication means (245) configured to receive said measured data and transmit data to at least one selected from the group consisting of a wireless module connection (244), a patient (242), a physician (246), a physiotherapist (246), and a dedicated web-based application (247).

Additionally, the system includes a dedicated web-based application (247) further configured to intercommunicating with said patient (242) or with Physiotherapist or Physician (246).

Cloud (249) and computer (250) are used to store data to store data, said data further includes input sensory data, recommendation/notification/hazards/maintenance, treatment plan etc.

FIG. 14 presents a method of conducting a remote medical examination of patient and joint. comprising steps of:
(a) Acquiring input (310) via a sensory array (243)
(b) Transferring the input data (312) from the sensory array (243) to a digital processor (241), via wireless means
(c) Transferring the data (311) from the digital processor (241) to storage via computer or cloud (312)
(d) Transferring the data (313) from the digital processor to a communication means (245) via wireless means
(e) Transferring the data (314) from the communication means (245) to a patient (242) via wireless means
(f) Transferring the data (315) from the communication means (245) to a physiotherapist/physician (246) via wireless means
(g) Transferring the data (316) from the communication means (245) to a dedicated web-based application (247) via wireless means
(h) Diagnosing the data (317) by the physiotherapist/physician (246)
(i) Feedback to patient (242) and to physiotherapist/physician (246) by means of the dedicated web-based application (247)
(j) Loading treatment plan (319) on cloud and download in real time—via wireless means and cloud
(k) Sending recommendation/notification/hazards/maintenance (320) by physiotherapist/physician (246) to patient (242) via wireless means and cloud
(l) Collecting data for personalized patient files and for general research by physiotherapist/physician (246) via computer and/or cloud
(m) Changing/modifying treatment (322) by physiotherapist/physician (246) via wireless and cloud.

According to embodiments of the invention, the brace may be used for:
control, guide, limit and/or immobilize an extremity, joint or body segment for a particular reason
restrict movement in a given direction;
assist movement generally;
reduce weight bearing forces for a particular purpose;
aid rehabilitation from fractures after the removal of a cast; and
to otherwise correct the shape and/or function of the body, to provide easier movement capability or reduce pain.

According to embodiments of the invention, the physiotherapy treatment, using the physiotherapeutic brace, adapted to:
reduce pain and inflammation;
normalize the joint range of motion;
strengthen the knee: Quadriceps and Hamstrings;
strengthen the lower limb: Calves, Hip and Pelvis muscles;
improve patellofemoral (knee cap) alignment;
normalize the muscle lengths;
improve the proprioception, agility and balance;
improve technique and function: walking, running, squatting, hopping and landing; and
minimize the chance of repeat injury.

It will be appreciated by persons skilled in the art that embodiment of the invention are not limited by what has been particularly shown and described hereinabove. Rather the scope of at least one embodiment of the invention is defined by the claims below.

1.-120. (canceled)

121. A method for providing exercise to at least one muscle connected to, or otherwise affecting the function of, a joint; said method comprising steps of:
a. providing a modular brace; said modular brace comprises:
i. lateral and medial rigid articulated cages;
ii. lateral and medial replaceable elastic members; and
iii. at least one joint-covering, replaceable elastic member, having a central opening;
b. connecting each of said lateral and medial replaceable elastic members to end portions of said lateral and medial rigid articulated cages, respectively; and
c. accommodating said at least one joint-covering, replaceable elastic member on said joint and connecting to both said lateral and medial rigid articulated cages;
thereby producing predetermined resistance force against muscles of said limb, while restricting said joint's motion to its normal articulation;
thereby simultaneously protecting said joint from external insult and preventing pressure on the same by said at least one joint-covering, replaceable elastic member.

122. The method of claim 121, additionally comprising the step of mounting said modular brace on a joint selected from the group consisting of knee, elbow or ankle.

123. The method of claim 121, additionally comprising the step of configuring said lateral and medial replaceable elastic members to each have a two-fold symmetrical axis.

124. The method of claim 121, additionally comprising the step of configuring said at least one joint-covering, replaceable elastic member to have a four-fold symmetrical axis.

125. The modular brace of claim 121, wherein dimensions of said central opening are configured for fixation of said joint.

126. The modular brace of claim 121, wherein said lateral and medial replaceable elastic member are adjustable.

127. The modular brace of claim 121, further comprising adjustable securing elements, configured for mounting said brace on said joint.

128. A set of replaceable elastic members for interconnecting lateral and medial rigid articulated cages of a modular brace which is mountable on a joint and configured for exercising at least one muscle connected to, or otherwise affecting the function of, said joint;
said replaceable elastic members comprise at least one selected from a group comprising: lateral, medial, and joint-covering;
said set comprise a plurality of elastic members selected from a group consisting of: at least two lateral elastic members, at least two medial elastic members, at least two joint-covering elastic members;
wherein said replaceable elastic members are configured to produce predetermined resistance force against muscles connected to said joint;
further wherein each of said components of said set is adapted to provide a different amount of said predetermined resistance force.

129. The set of claim 128, wherein said components of said set provide different amount of said predetermined resistance force by at least one selected from a group consisting of: differing in elasticity and differing in size.

130. The set of claim 128, wherein said lateral and medial cages are configured for restriction of said joint's motion to its normal articulation.

131. The set according to claim 128, wherein each of said elastic members have a color according to said amount of predetermined resistance force provided by them.

132. The set of claim 128, wherein said lateral and said medial replaceable elastic members, are adapted each to be connected to end portions of said lateral and medial rigid articulated cages, respectively.

133. The set of claim 128, wherein said at least one joint-covering, replaceable elastic member, has a central opening configured for accommodating said joint.

134. The set of claim 128, wherein said at least one joint-covering, replaceable elastic member is connected to both said lateral and medial rigid articulated cages.

135. The set of claim 128, wherein said at least one joint-covering, replaceable elastic member is adapted to simultaneously protect said joint from external insult while preventing pressure on the same.

136. The set of claim 128, wherein said replaceable elastic members are made of a material selected from a group consisting of: natural rubber, synthetic rubber, silicone rubber, latex, mesh, web, and any combination thereof.

137. A modular ankle brace, mountable on a foot configured for exercising at least one muscle connected to an ankle joint, comprising:
a. lateral and medial rigid articulated cages having each a central opening configured for accommodation on said ankle;
b. lateral and medial replaceable elastic members, each connected to end portions of said lateral and medial rigid articulated cages, respectively;
c. a rear replaceable elastic member, configured for accommodation on a heel, connected to both said lateral and medial rigid articulated cages;
wherein said lateral, medial and rear replaceable elastic members are configured to produce predetermined resistance force against muscles connected to said ankle, while said lateral and medial cages are configured for restriction of said ankle motion to its normal articulation;
further wherein said lateral and medial rigid articulated cages are adapted to simultaneously protect said ankle from external insult while preventing pressure on the same.

138. The modular ankle brace according to claim 137, wherein said lateral and said medial replaceable elastic members, are adapted each to be connected to end portions of said lateral and medial rigid articulated cages, respectively.

139. The modular ankle brace according to claim 137, wherein said rear replaceable elastic member, has a central opening configured for accommodating the heel.

140. The modular ankle brace according to claim 137, wherein said rear replaceable elastic member is connected to both said lateral and medial rigid articulated
